(19) United States
(12) Patent Application Publication
(10) Pub. No.: US 2016/0183880 A1
(43) Pub. Date: Jun. 30, 2016

(54) BIOLOGICAL INFORMATION MEASURING DEVICE AND DRIVING CONTROL METHOD OF THE SAME

(71) Applicant: CASIO COMPUTER CO., LTD., Tokyo (JP)

(72) Inventor: Hideo Abe, Tokorozawa-shi (JP)

(73) Assignee: CASIO COMPUTER CO., LTD., Tokyo (JP)

(21) Appl. No.: 14/980,921

(22) Filed: Dec. 28, 2015

(30) Foreign Application Priority Data
Dec. 26, 2014 (JP) 2014-264265

(51) Int. Cl. A61B 5/00; A61B 5/0205

(52) U.S. Cl. CPC A61B 5/721; A61B 5/0205; A61B 5/725; A61B 5/7285; A61B 5/742; A61B 5/02416

(57) ABSTRACT

A biological information measuring device includes: a biological information measuring unit for measuring biological information of a user; a motion signal output unit for outputting a motion signal corresponding to motion of the user; and a control unit.

The control unit causes the biological information measuring unit to measure the biological information when amplitude of the motion signal is smaller than a first setting value during a first period and causes the biological information measuring unit to stop measurement when the amplitude is greater than the first setting value during a second period and there is a period where the amplitude is greater than a second setting value, which is greater than the first setting value, in the second period.

### CROSS-REFERENCE TO RELATED APPLICATIONS

A corresponding Japanese application is Japanese Patent Application No. 2014-264265, filed on Dec. 26, 2014.

### BACKGROUND OF THE INVENTION

1. Field of the Invention

The present invention relates to a biological information measuring device and a driving control method of the biological information measuring device.

2. Description of the Related Art

As a casual tool for managing health by an individual, for example, a wrist watch mounted with a pulse wave sensor is expected to enable easy pulse wave measurement on a daily basis.

However, when power consumed by the pulse wave sensor is high, a battery life becomes short and thus battery replacement or charging is frequently required, which is inconvenient.

Therefore, in a device having such a pulse wave sensor, it is desired to save power as much as possible while pulse wave measurement is enabled.

Here, it is known that a pulse wave signal, output from the pulse wave sensor while a human body is in motion, includes noise attributable to human body motion. In this case, a technique is known for removing a noise component attributable to the human body motion from the pulse wave signal by filtering. For example, Japanese Laid-Open Patent Publication No. 2012-179209 discloses a technique for changing filter processing to be applied to a pulse wave signal when there is motion and when there is no motion in order to effectively address noise.

However, according to the technique disclosed in Japanese Laid-Open Patent Publication No. 2012-179209, the pulse wave sensor is in operation at all times and is always performing pulse wave measurement. Moreover, filter processing including a relatively large volume of calculation such as frequency analysis for removal of motion component included in the pulse wave signal is also performed at all times. Thus, there has been an issue that such circumstances result in increased power consumption.

### BRIEF SUMMARY OF THE INVENTION

The present invention has an advantage of providing a biological information measuring device including a biological information measuring unit for enabling biological information measurement while saving power and a driving control method of a biological information measuring device.

According to an embodiment of the present invention, there is provided a biological information measuring device, including: a biological information measuring unit configured to measure biological information of a user; a motion signal output unit configured to output a motion signal corresponding to motion of the user; and a control unit, wherein the control unit causes the biological information measuring unit to measure the biological information when amplitude of the motion signal is smaller than a first setting value during a first period, and wherein the control unit causes the biological information measuring unit to stop measurement of the biological information when the amplitude of the motion signal is greater than the first setting value during a second period and when there is a period where the amplitude of the motion signal is greater than a second setting value, which is greater than the first setting value, in the second period.

According to another embodiment of the present invention, there is provided a biological information measuring device, including: a biological information measuring unit configured to measure biological information of a user; a motion signal output unit configured to output a motion signal corresponding to motion of the user; and a control unit configured to control the biological information measuring unit and the motion signal output unit, wherein the control unit determines that the user is in a motion state when amplitude of the motion signal is greater than a first setting value during a predetermined period which includes a predetermined time length, the control unit causes the biological information measuring unit to stop measurement of the biological information when it is determined that the user is in the motion state and the amplitude of the motion signal is greater than a second setting value, which is greater than the first setting value, has existed in the predetermined period, and the control unit controls the biological information measuring unit to measure the biological information when it is determined that the user is in the motion state; the amplitude of the motion signal during the predetermined period is smaller than the second setting value; and the motion signal during the predetermined period has a predetermined periodicity.

According to another embodiment of the present invention, there is provided a driving control method of a biological information measuring device, wherein the biological information measuring device includes a biological information measuring unit configured to measure biological information of a user, the driving control method including the steps of: causing the biological information measuring unit to measure biological information of the user when amplitude of a motion signal corresponding to motion of the user is smaller than a first setting value during a first period; and causing the biological information measuring unit to stop measurement of the biological information when the amplitude of the motion signal is greater than the first setting value during a second period and when there is a period where the amplitude of the motion signal is greater than a second setting value, which is greater than the first setting value, in the second period.

### BRIEF DESCRIPTION OF THE SEVERAL VIEWS OF THE DRAWING

FIG. 1 is a block diagram illustrating a configuration of a pulse wave measuring device according to an embodiment of the invention;

FIG. 2 is a flowchart illustrating processing operations of a control unit of a first example;

FIG. 3 is a flowchart illustrating detailed processing of determining a non motion state in FIG. 2;

FIG. 4 is a flowchart illustrating detailed processing of determining whether removal of motion is possible in FIG. 2;

FIGS. 5A and 5B are timing charts illustrating an operation state of the first example;

FIG. 6 is a conceptual diagram of a system illustrating a system configuration and schematic operations with connection to a health care server;

FIG. 7 is a flowchart illustrating processing operations of the health care server in FIG. 6;

FIG. 8 is a flowchart illustrating processing operations of a control unit of a second example;

FIGS. 9A and 9B are timing charts illustrating an operation state of the second example;

FIG. 10 is a flowchart illustrating processing operations of a control unit of a third example;

FIGS. 11A and 11B are timing charts illustrating operations of the third example; and

FIG. 12 is a diagram illustrating overall processing of removing a motion component by filtering.

### DETAILED DESCRIPTION OF THE INVENTION

Embodiments of a biological information measuring device according to the present invention will be described below in detail with reference to the drawings.

(Configuration of an Embodiment)

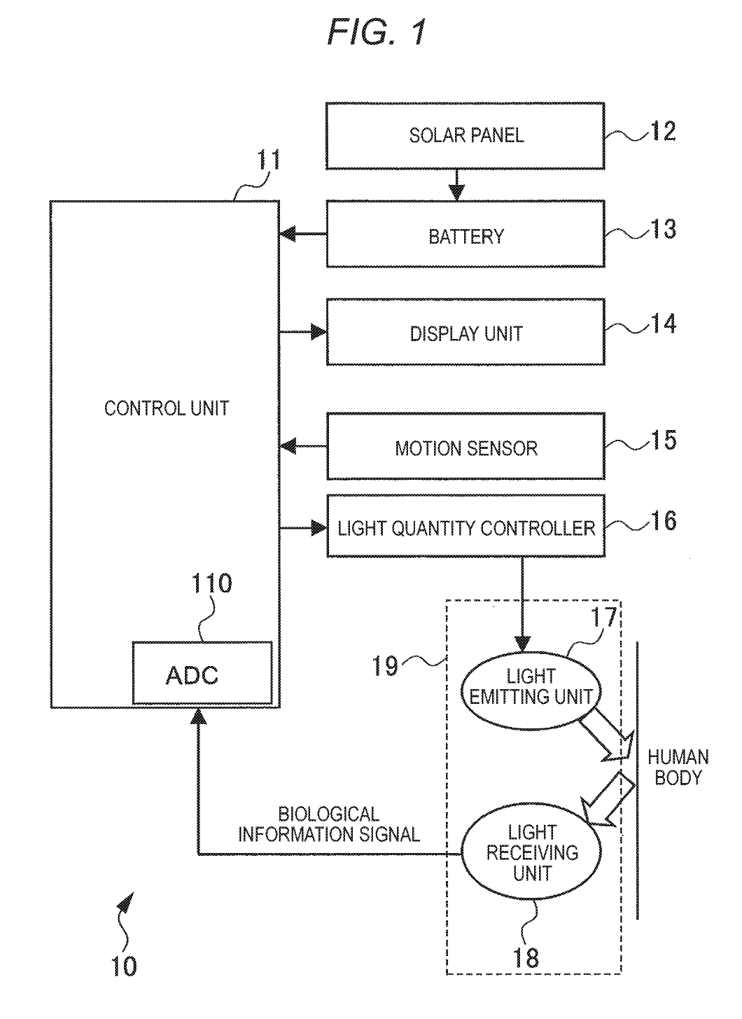

FIG. 1 is a block diagram illustrating a configuration of a biological information measuring device according to the present embodiment.

As illustrated in FIG. 1, a biological information measuring device 10 according to the present embodiment measures a pulse rate or the like from a biological information signal.

In the present embodiment, a case will be described where a biological information sensor is a pulse wave sensor that measures a pulse wave in human blood vessels as in the following. However, the biological information sensor is not limited to a pulse wave sensor that measures a pulse wave in human blood vessels. For example, the biological information sensor may measure any biological information that is influenced by human body motion such as a heart rate, blood-pressure value, respiratory rate, etc. of a human body.

The biological information measuring device 10 includes a control unit 11 as the center of control, a solar panel 12 and a battery 13 as a source of power supply, a display unit 14 for displaying data generated by the control unit 11, a motion sensor (motion signal output unit) 15 as a detection unit of motion, a light quantity controller 16, a light emitting diode (LED) 17 as a light emitting unit, and a photo detector (PD) 18 as a light receiving unit.

Here, the LED 17 and PD 18 form a photoelectric pulse wave sensor (biological information measuring unit) 19 for measuring and acquiring a pulse wave corresponding to pulses of a human body.

The control unit 11 includes a central processing unit (CPU) and further includes an analog digital convertor (ADC) 110 which is a conversion unit for converting an analog signal into a digital signal by importing the analog signal at a timing corresponding to a predetermined sampling frequency.

The control unit 11 controls the respective blocks (the solar panel 12, battery 13, display unit 14, motion sensor 15, light quantity controller 16, LED 17, and light receiving unit 18) externally connected thereto according to, for example, a program stored in a memory included therein and also performs data processing for calculation of a pulse rate or the like.

The solar panel 12 generates power from the sunlight and charges the battery 13 under control by the control unit 11 by being connected to the battery 13, the source of power supply.

The display unit 14 includes a display device such as a liquid crystal display device (LCD) and displays, for example, data generated by the control unit 11 on the display device.

The motion sensor 15 detects a change in acceleration corresponding to motion of a living body of the user and outputs an acceleration signal (motion signal) and is exemplified by a three-axis acceleration sensor.

The light quantity controller 16 controls a current flowing in the LED 17, thereby performing luminance control of the LED 17.

The LED 17 forming the pulse wave sensor 19 irradiates a human body (skin) with light. The PD 18 forming the pulse wave sensor 19 receives reflection light reflected by the human body (skin) after irradiation to the human body (skin) from the LED 17 and photoelectrically converts the received reflection light.

Thereafter, a pulse wave signal (biological information signal) photoelectrically converted and generated by the PD 18 is input to the ADC 110 of the control unit 11, converted into a digital value by the ADC 110, and imported to the control unit 11.

The light irradiated to the human skin from the LED 17 reaches a surface or an inner part of the skin, where a part of the light is reflected while another part is absorbed. In a blood vessel in the body, light is absorbed by hemoglobin. An amount of hemoglobin is dependent on an amount of blood flow. Based on this principal, the control unit 11 detects, via the PD 18, a change in the reflection light reflected by the human body (skin) after being irradiated from the LED 17 and thereby observes a pulse wave in the blood vessel of the human body.

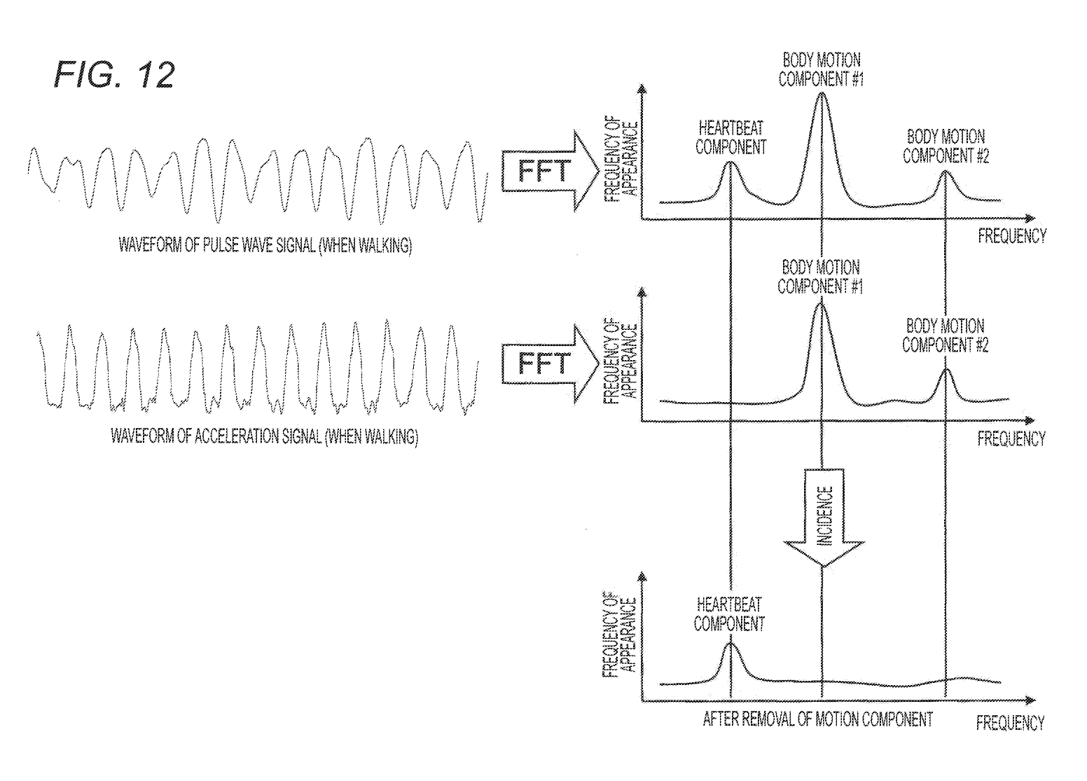

FIG. 12 is a diagram illustrating overall processing of removing a motion component from a pulse wave signal.

As illustrated in FIG. 12, a waveform of a pulse wave signal (pulse wave signal waveform) measured when a user is in motion such as walking or running is superimposed with a motion component attributable to the motion. To extract a pulse component from the pulse wave signal waveform superimposed with this motion component, processing for removing the motion component (motion removing filter processing) is preferable.

In order to perform this motion removing filter processing, first, a frequency component of the acceleration signal (motion signal) and pulse wave signal output from the motion sensor 15 is analyzed (e.g. Fourier transform), thereby examining a distribution of the frequency component for both signal waveforms.

Thereafter, based on comparison between the frequency distribution of the acceleration signal and the frequency distribution of the pulse wave signal, a frequency component estimated as motion component (in FIG. 12, motion components #1 and #2) is extracted.

Furthermore, to remove the frequency component estimated as the motion component from the pulse wave signal, calculation processing is performed such that a difference between the two is obtained. In this manner, the motion removing filter processing includes a relatively large calculation volume and thus relatively high power is consumed.

Configurations of respective examples of the present embodiment will be described below.

The control unit 11 determines whether a user is in a motion state where the user is in motion or the user is in a non motion state where the user is substantially in no motion according to a level of motion detected by the motion sensor 15.

The control unit 11 further controls execution of pulse wave measurement and execution of the motion removing filter processing of the pulse wave signal according to a level of motion when the user is in the motion state.

The control unit 11, when determining that the user is in the non motion state, causes the pulse wave sensor to perform pulse wave measurement and controls not to perform the motion removing filter processing to the pulse wave signal (first example). This is because the motion removing filter processing is not required since substantially no motion component is superimposed to the pulse wave signal in such a state.

The control unit 11, when determining that the user is in the motion state and that a level of motion is greater than a predetermined value, namely, the user is in relatively big motions, controls not to perform pulse wave measurement. This is because a number of motion components are superimposed to the pulse wave signal in such a state and thus a volume of calculation processing in the motion removing filter processing increases as well as power consumption required therefor. Furthermore, even when the motion removing filter processing is performed, it is difficult to remove those motion components from the pulse wave signal in a preferable manner and thus reliability of pulse wave measurement is degraded.

When the control unit 11 determines that: the user is in the motion state; a level of motion is lower than the predetermined value; and the motion signal has predetermined periodicity, namely, when an amount of variations in the motion signal within a frequency range thereof in a predetermined period of time is lower than an allowable value, the control unit 11 causes the pulse wave sensor to perform pulse wave measurement and controls to perform the motion removing filter processing to the pulse wave signal. This is because, although the pulse wave signal is superimposed with the motion component in such a state, this motion component is relatively small and has periodicity. Therefore, a volume of calculation processing in the motion removing filter processing for removing this motion component is relatively low and thus power consumption required therefor is relatively low. Furthermore, the motion removing filter processing allows for removing the motion component in a relatively preferable manner and thus reliability of pulse wave measurement is relatively enhanced.

The control unit 11 may further control to change a sampling frequency in the ADC 110 according to the motion state or the non motion state having been determined (second example).

That is, when determining that the user is in the non motion state, the control unit 11 sets a sampling frequency to a low sampling frequency with a relatively low frequency and controls not to perform the motion removing filter processing. This is because substantially no motion component is superimposed to the pulse wave signal in such a state and thus it is estimated that the pulse wave signal includes substantially no frequency component that is relatively high and attributable to motion.

On the other hand, when it is determined that: the user is in the motion state; a level of motion is lower than the predetermined value; and the motion signal has predetermined periodicity, the control unit 11 sets a sampling frequency to a high sampling frequency with a relatively high frequency and controls to perform the motion removing filter processing. This is because it is estimated that motion component is superimposed to the pulse wave signal in such a state and thus the pulse wave signal also includes a relatively high frequency component attributable to motion.

The control unit 11 may further control to change operations of the respective units to one of an interval measurement mode and a continuous measurement mode according to the motion state or the non motion state having been determined (third example).

That is, when determining that the user is in the non motion state, the control unit 11 sets operations of the respective units to the interval measurement mode and controls not to perform the motion removing filter processing. This is because it is estimated that the user is in a relatively still state with substantially no motion and thus variations in pulse rate over time are relatively small.

On the other hand, when it is determined that: the user is in the motion state; a level of motion is lower than or equal to the predetermined value; and the motion signal has predetermined periodicity, the control unit 11 sets operations of the respective units to the continuous measurement mode and controls to perform the motion removing filter processing. This is because it is estimated that the user is in some motion and thus variations in pulse rate over time are relatively large.

(Operations in an Embodiment)

Operations of a pulse wave measuring device (biological information measuring device) 10 according to the present embodiment will be described below in detail for each of the examples.

### FIRST EXAMPLE

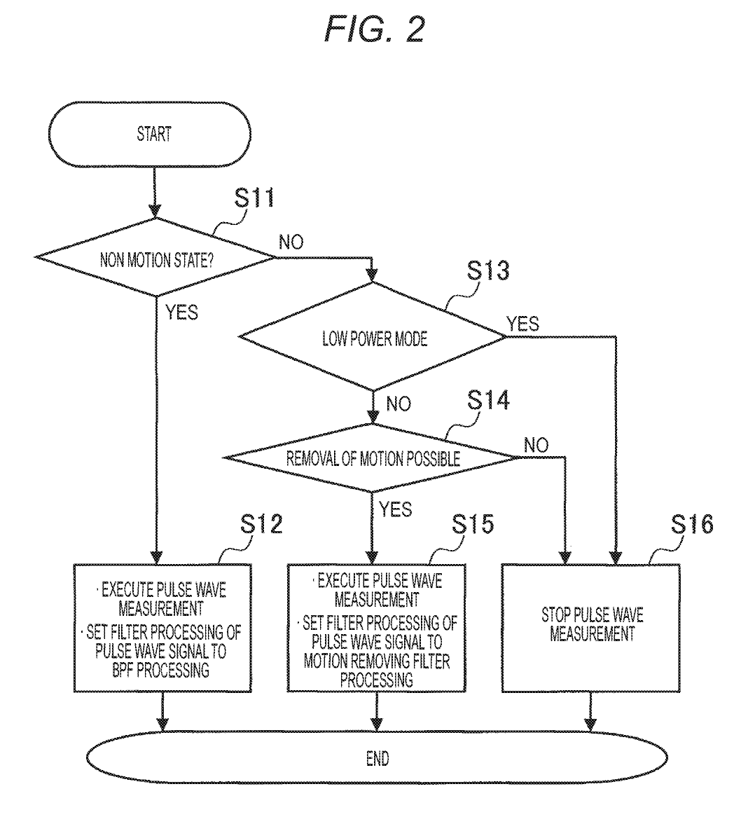

First, operations in the first example will be described with reference to FIGS. 2 to 5.

In a flowchart in FIG. 2, the control unit 11 first determines whether the user is in the non motion state (step S11).

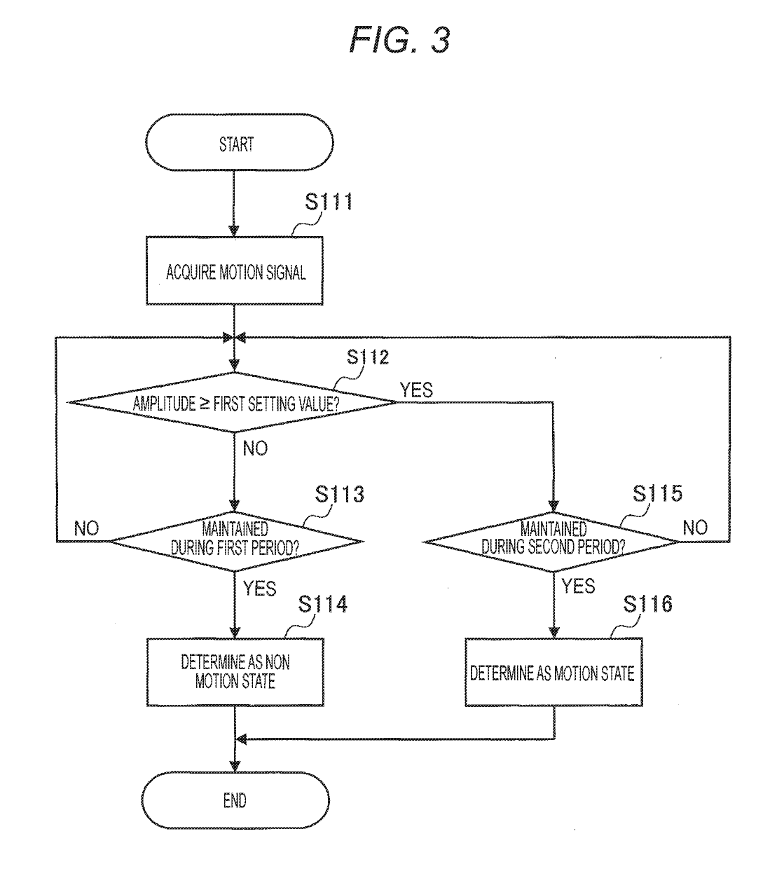

Details of this processing for determining the non motion state is illustrated in FIG. 3.

According to FIG. 3, the control unit 11 first imports an acceleration signal (motion signal) output from the motion sensor 15 (step S111).

Next, the control unit 11 determines whether there is motion based on the imported acceleration signal (step S112). This determination on motion is performed by comparing amplitude of the acceleration signal and a first setting value that is a threshold for determination.

Here, when the control unit 11 determines that the amplitude of the acceleration signal is lower than the first setting value (NO in step S112), the control unit 11 then determines whether the aforementioned state where the amplitude of the acceleration signal is lower than the first setting value has been maintained during a first period including a predetermined first time length (step S113).

When determining that the aforementioned state has been maintained during the first period (YES in step S113), the control unit 11 determines that the user is in the non motion state (step S114).

On the other hand, when determining that the aforementioned state has not been maintained during the first period (NO in step S113), the control unit 11 does not determine that the user is in the non motion state and the process returns to the determination processing of amplitude of step S112.

On the other hand, when the control unit 11 determines that the amplitude is greater than or equal to the first setting value in the determination processing of motion of step S112 (YES in step S112), the control unit 11 then determines whether the aforementioned state has been maintained during a second period including a predetermined second time length (step S115).

When determining that the aforementioned state has been maintained during the second period (YES in step S115), the control unit 11 determines that the user is in the motion state (step S116).

On the other hand, when determining that the aforementioned state has not been maintained during the second period (NO in step S115), the control unit 11 does not determine that the user is in the motion state and the process returns to the determination processing of amplitude of step S112.

Here, the aforementioned first time length and the second time length are set to, for example, two to ten seconds. The first time length and second time length may be the same or different.

That is, the control unit 11 does not determine that the user is in the non motion state when the state where the amplitude of the acceleration signal is smaller than the first setting value has been maintained only temporally for a short period of time but determines that the user is in the non motion state when this state has been maintained during a certain period of time. On the other hand, the control unit 11 does not determine that the user is in the motion state when the state where the amplitude of the acceleration signal is greater than the first setting value has been maintained only temporally for a short period of time but determines that the user is in the motion state when this state has been maintained during a certain period of time.

Now, referring back to FIG. 2, when determining that the user is in the non motion state in the determination processing of non motion state of step S11 (Yes in step S11), the control unit 11 causes the pulse wave sensor 19 to execute pulse wave measurement. The control unit 11 further determines that the motion removing filter processing for removing motion component is not required for the pulse wave signal output from the pulse wave sensor 19. This is because substantially no motion component is superimposed to the pulse wave signal in the non motion state.

Thereafter, the control unit 11 sets the filter processing for the pulse wave signal to a band pass filter (BPF) processing including a relatively small volume of calculation and performs the BPF processing (step S12).

Note that, here, this BPF processing may not be performed to the pulse wave signal. The filter processing itself may also be omitted.

On the other hand, when determining that the user is in the motion state in the determination processing of the non motion state of step S11 (NO in step S11), the control unit 11 then determines whether an operation mode of the pulse wave measuring device 10 is set to a low power mode (step S13).

Here, the low power mode allows for operation in a power saving mode with more priority on a battery life and may be, for example, manually set in advance.

When determining that the operation mode is not set to the low power mode (NO in step S13), the control unit 11 then determines whether removal of motion is possible (step S14).

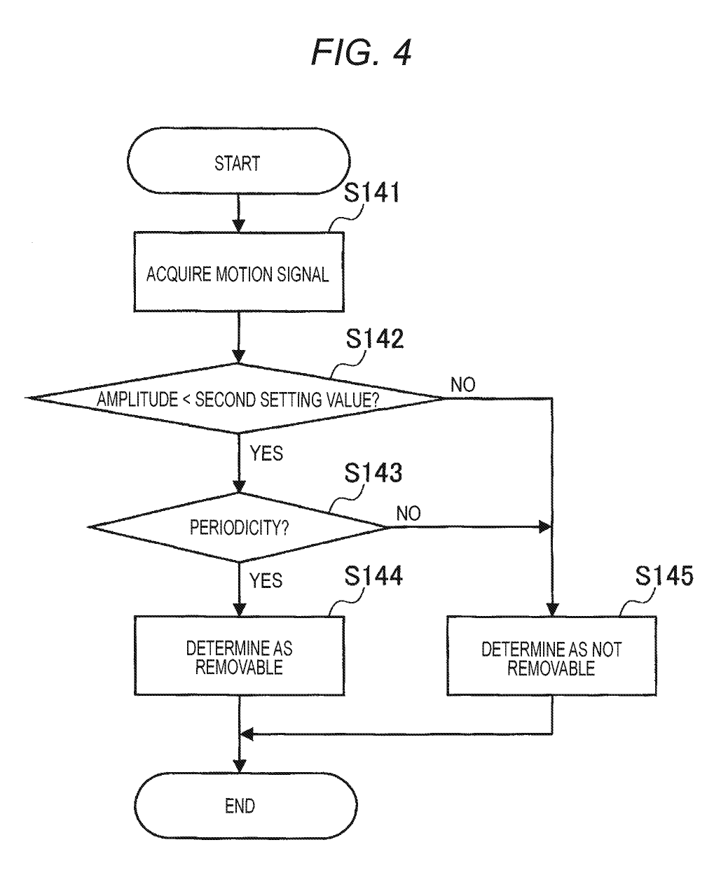

Details of this processing for determining whether removal of motion is possible is illustrated in FIG. 4.

According to FIG. 4, the control unit 11 first imports an acceleration signal (motion signal) output from the motion sensor 15 (step S141).

Next, the control unit 11 determines a level of the motion during the second period based on the imported acceleration signal (step S142). This determination on the level of motion is performed by comparing amplitude of the acceleration signal and a second setting value that is a threshold for this determination.

This second setting value is set to a value greater than the first setting value. Here, when the control unit 11 determines that the amplitude of the acceleration signal during the second period is lower than the second setting value (YES in step S142), the control unit 11 then determines whether the acceleration signal has predetermined periodicity (step S143).

For determination on periodicity, specifically, the control unit 11 for example obtains a frequency range of the acceleration signal and determines whether an amount of variation within this frequency range in a predetermined time is greater than an allowable value. When this amount of variation within the frequency range is smaller than the allowable value, it is determined that there is periodicity.

For determination on periodicity, when the amount of variation within the frequency range of the acceleration signal is greater than the allowable value and when the acceleration signal varies over time in a relatively random manner, a volume of calculation increases in calculation processing for removing a motion component corresponding to the acceleration signal from the pulse wave signal and thus power consumption required therefor increases. Furthermore, it is difficult to remove the motion component from the pulse wave signal in a preferable manner and thus reliability of pulse wave measurement is degraded.

Therefore, the control unit 11 determines that removal of the motion component is impossible when there is no periodicity and controls not to perform pulse wave measurement.

When determining that the acceleration signal has periodicity (YES in step S143), the control unit 11 determines that the motion component is removable (step S144).

On the other hand, when determining that the acceleration signal has no periodicity (NO in step S143) or when determining, in the determination processing of amplitude value comparison of step S142, that there is a period where the amplitude of the acceleration signal is greater than or equal to the second setting value during the second period (NO in step S142), the control unit 11 determines that the motion component is not removable (step S145).

Now, referring back to FIG. 2, when determining that removal of motion is possible in the determination processing of removing motion of step S14 (YES in step S14), the control unit 11 causes the pulse wave sensor 19 to execute pulse wave measurement. The control unit 11 further sets filter processing of the pulse wave signal output from the pulse wave sensor 19 to the motion removing filter processing and performs the motion removing filter processing (step S15).

That is, the control unit 11 performs the motion removing filter processing for removing the motion component in order to extract a pulse component from the pulse wave signal waveform superimposed with the motion component.

As described above with reference to FIG. 12, this motion removing filter processing is performed by analysis of frequency components of a waveform of the acceleration signal output from the motion sensor 15 and a waveform of the pulse wave signal as well as calculation processing for removing the motion component from the pulse wave signal.

Note that, when determining that the low power mode is set in the determination processing of low power mode of step S13 (YES in step S13) and when determining that removal of motion is impossible in the determination processing of removing motion of step S14 (NO in step S14), the control unit 11 turns off the LED 17 (to stop irradiation with light) and stops operation of the pulse wave sensor 19, thereby stopping pulse wave measurement (step S16).

Here, operation of the PD 18 may further be stopped.

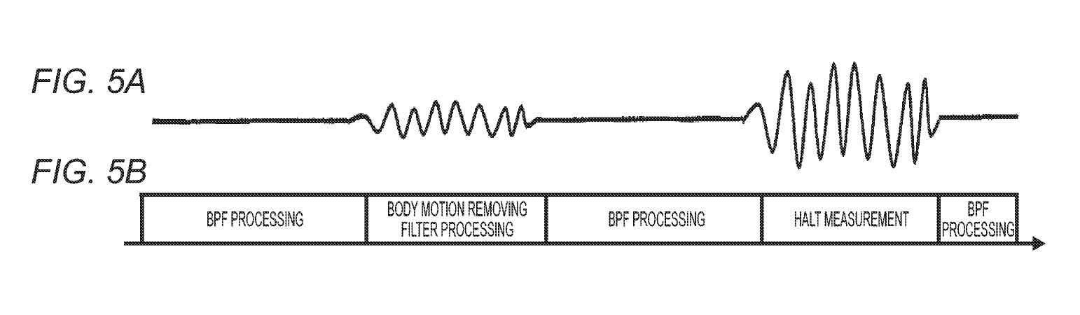

FIGS. 5A and 5B are timing charts illustrating an operation state of the first example.

In FIG. 5A, an acceleration signal (motion data) output from the motion sensor 15 is illustrated. In FIG. 5B, a filter type to be applied to a pulse wave signal selected by the control unit 11 is illustrated.

According to the first example, the control unit 11 determines whether there is motion from data acquired from the motion sensor 15. When the motion is greater than or equal to a setting value the control unit 11 selects the body motion removing filter processing. When the motion is smaller than the setting value the control unit 11 selects the BPF processing. This allows for pulse wave measurement with the least volume of calculation.

When the motion is small, the BPF processing requiring no frequency analysis with relatively high power consumption is applied and thus power can be saved.

Note that, other than selecting a type of filter processing, changing settings of a parameter for the filter processing also results in a similar effect. Furthermore, a filter processing that can replace the BPF processing may be used. Processing can be performed without a filter processing.

Note that the low power mode allows for controlling operations such that pulse wave measurement is performed only in the non motion state and that pulse wave measurement is not performed in the motion state with more priority on saving power.

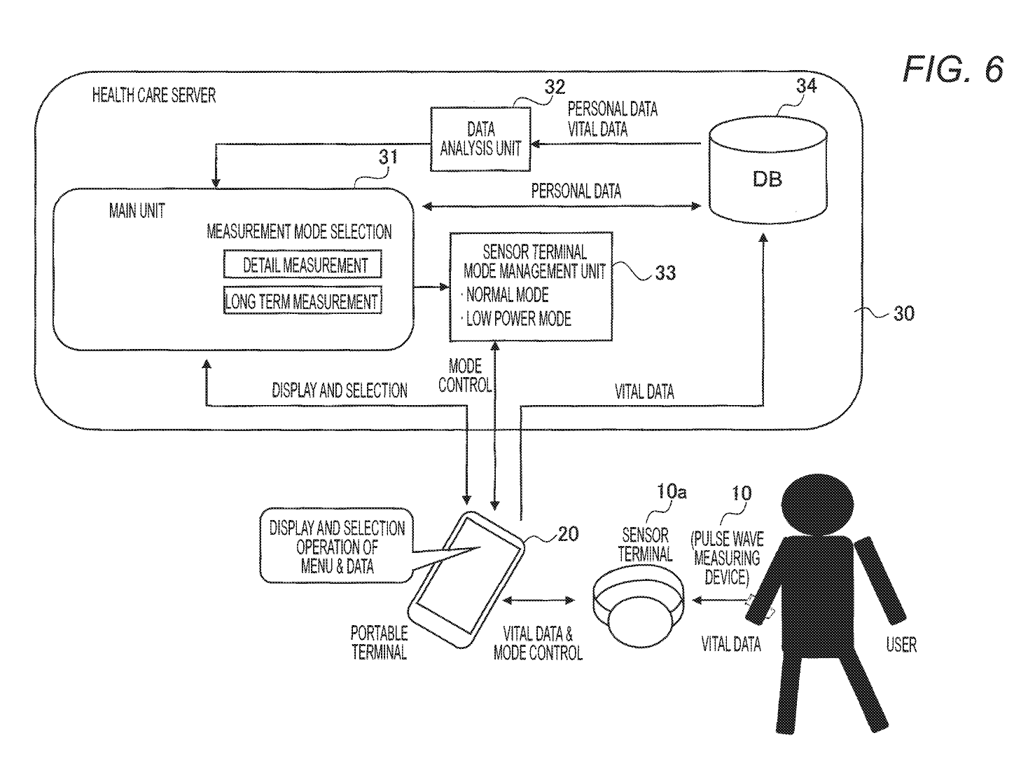

Switching between the low power mode and a normal mode that is not the low power mode may be manually performed by a user according to a purpose. Alternatively, the control unit 11 or for example a health care server 30 in a cloud environment as illustrated in FIG. 6 may automatically switch between the low power mode and the normal mode according to circumstances.

FIG. 6 illustrates a configuration of a system for performing health management of an individual using the aforementioned pulse wave measuring device 10 and the health care server 30 in a cloud and overall operations thereof.

As illustrated in FIG. 6, a user (subject) wears on the arm the pulse wave measuring device 10 mounted with a sensor terminal 10a of a wrist-watch shape. The pulse wave measuring device 10 is connected to the health care server 30, for example by wireless communication, via a portable terminal 20 such as a smartphone and a network.

The health care server 30 includes a main unit 31, a data analysis unit 32, a sensor terminal mode management unit 33, and a data base 34.

A user requests, regularly or irregularly, the health care server 30 to analyze vital data including a pulse wave by selecting on a menu screen displayed on the portable terminal 20. For this end, the sensor terminal 10a occasionally transmits vital data measured.

The main unit 31 produces a measurement schedule upon a user's request and determines a measurement mode including the “detail measurement” and “long term measurement” based on the schedule.

Here, an operation mode of the pulse wave measuring device 10 is set to the low power mode or the normal mode.

The operation mode set here is notified to the sensor terminal 10a via the network under control by the sensor terminal mode management unit 33. The sensor terminal 10a collects vital data according to the notified mode.

The collected vital data is accumulated in the data base 34 of the health care server 30.

The data analysis unit 32 analyzes personal data accumulated in the data base 34 and vital data for a predetermined period according to the measurement mode of the health care server 30. The health care server 30 then transmits the result to the requesting portable terminal 20 via the network.

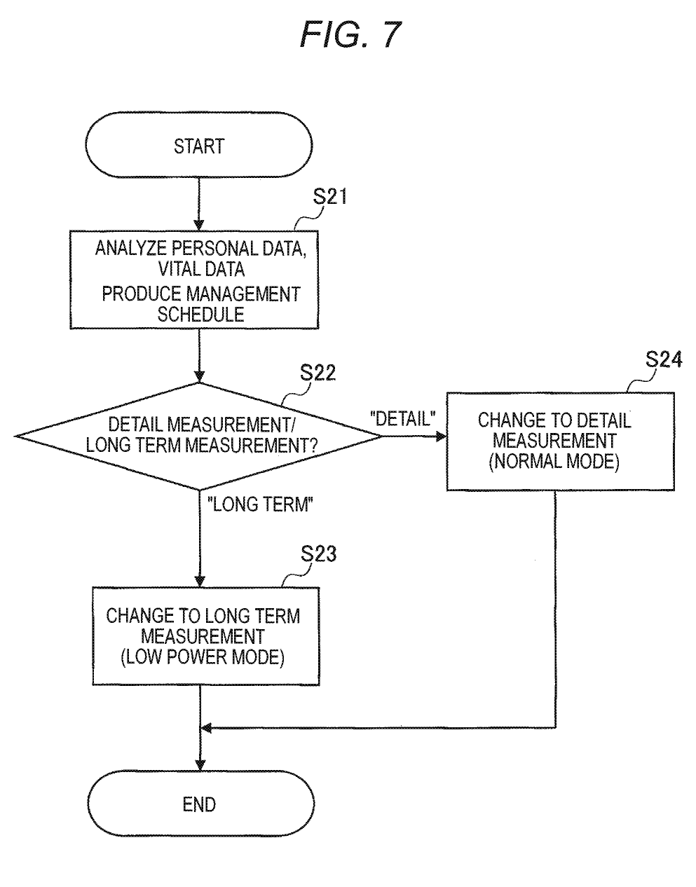

FIG. 7 is a flowchart illustrating processing procedure of the health care server 30.

According to FIG. 7, in the health care server 30, the data analysis unit 32 analyzes received personal data and vital data and produces a management schedule under control by the main unit 31 (step S21).

Here, the main unit 31 discriminates between the detail measurement mode and long term measurement mode from the produced management schedule (step S22).

The main unit 31 executes processing for changing to the low power mode (step S23) in the long term measurement mode (“long term” in step S22) and executes processing for changing to the normal mode (step S24) in the detail measurement mode (“detail” in step S22).

Here, the sensor terminal mode management unit 33 transmits a mode to be set to the sensor terminal 10a via the network and the portable terminal 20 and instructs to measure vital data in that mode.

According to the pulse wave measuring device 10 of the first example, changing execution of the motion removing filter processing required for pulse rate calculation according to motion of the user allows for suppressing an increase in power consumption due to filter processing calculation as much as possible, thereby contributing to power saving.

The user can obtain analysis result based on vital data only by wearing on the arm the pulse wave measuring device 10 mounted with the sensor terminal 10a of a wrist-watch shape and being connected to the health care server 30 via the portable terminal 20 such as a smartphone and a network. This allows for mitigating a burden of health management.

### SECOND EXAMPLE

In the configuration where, in the pulse wave sensor 19, reflection light of light irradiated from the LED 17 is received by the PD 18 and a voltage output waveform thereof is converted into a digital value by the ADC 110 included in the control unit 11, a sampling frequency for the voltage output waveform in the ADC 110 is preferably higher than a frequency twice the highest frequency included in the voltage output waveform.

When there is motion, the pulse wave signal includes a relatively low frequency component attributable to pulses and a relatively high frequency component attributable to the motion.

Therefore, upon setting a sampling frequency when there is motion, the sampling frequency is preferably set to a high frequency (first frequency) where the motion is considered.

The pulse wave signal includes a waveform component attributable to pulses and a waveform component attributable to motion. A spectrum of the motion component is dependent on the speed of motion of a human and thus a relatively high frequency component is also included in the spectrum.

Therefore, a sampling frequency has been set to a relatively high frequency in consideration of the motion component in the related art.

Here, power consumption of the ADC 110 varies according to a sampling frequency Fs. The higher the sampling frequency Fs is, the more power consumption is.

Therefore, in the second example described below, a configuration is employed where the sampling frequency of the ADC 110 is set to a relatively low frequency (second frequency) in the non motion state, thereby allowing for suppressing power consumption of the ADC 110.

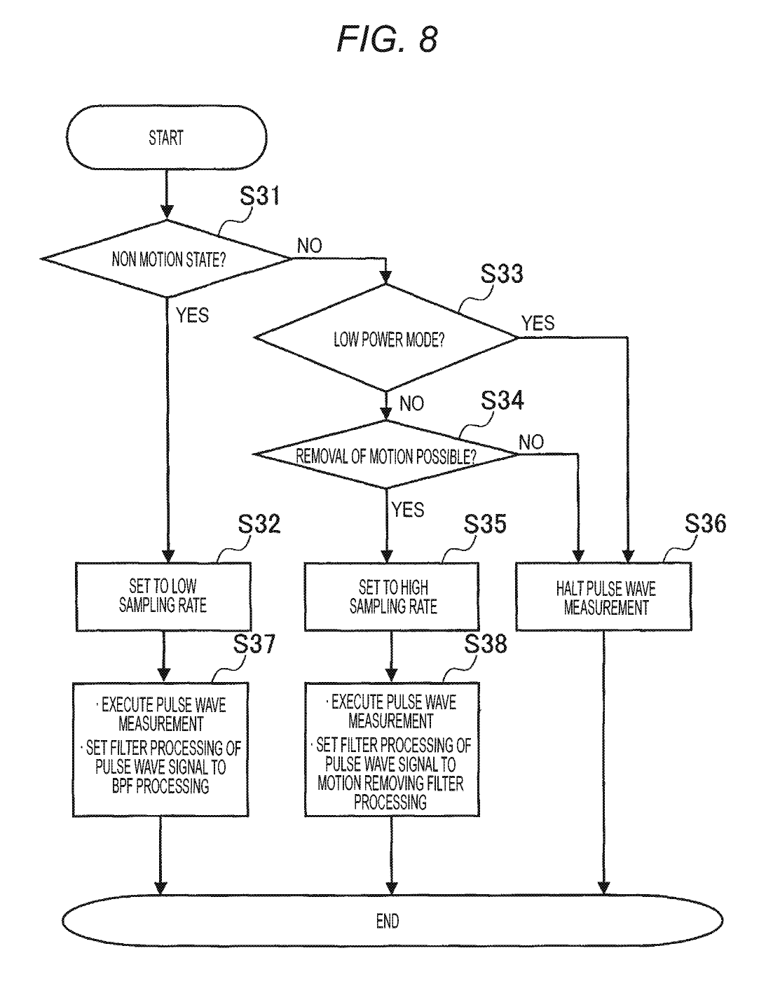

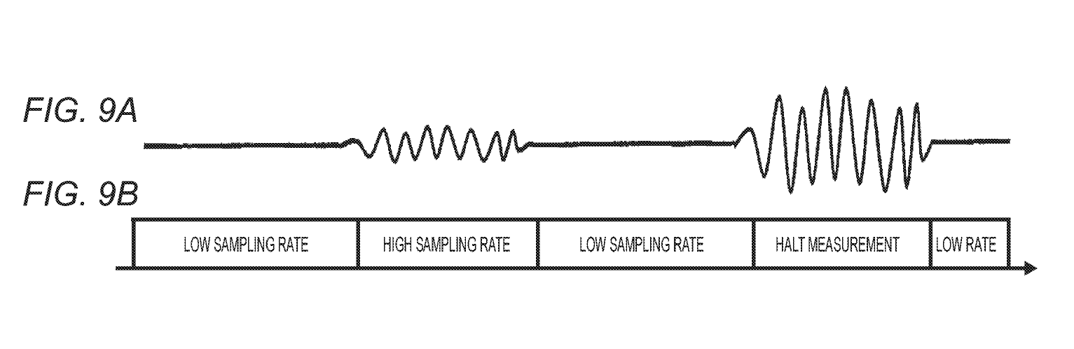

Operations in the second example will be described with reference to a flowchart in FIG. 8 and timing charts in FIGS. 9A and 9B.

In the flowchart in FIG. 8, the control unit 11 first determines whether the user is in the non motion state (step S31).

Details of the determination processing of non motion state are similar to the content described in the first example (FIG. 4) and thus description thereon is omitted to avoid redundancy.

Next, when determining that the user is in the non motion state (YES in step S31), the control unit 11 sets a sampling frequency of the ADC 110 to a relatively low sampling frequency (low sampling frequency) where only pulses are considered (step S32). This is because there is substantially no motion in the non motion state and the pulse wave signal includes substantially only the waveform component attributable to pulses. Therefore, the pulse wave signal input to the ADC 110 includes substantially only the relatively low frequency component attributable to pulses.

Next, the control unit 11 causes the pulse wave sensor 19 to execute pulse wave measurement. The control unit 11 further determines that the motion removing filter processing for removing motion component is not required for the pulse wave signal output from the pulse wave sensor 19 since substantially no motion component is superimposed to the pulse wave signal in the non motion state. Thereafter, the control unit 11 sets the filter for the pulse wave signal to the BPF including a relatively small volume of calculation and performs the BPF processing (step S37).

Note that, here, this BPF processing may not be performed to the pulse wave signal. The filter processing itself may also be omitted.

On the other hand, when determining that the user is in motion state (NO in step S31), the control unit 11 then determines whether an operation mode of the pulse wave measuring device 10 is set to the low power mode (step S33).

Here, as in the first example, the low power mode allows for operation in a power saving mode with more priority on a battery life and may be manually or automatically set in advance.

When determining that the operation mode is not set to the low power mode (NO in step S33), the control unit 11 then determines whether removal of motion is possible (step S34).

The determination processing of whether removal of motion is possible is similar to the content described in the first example (FIGS. 5A and 5B) and thus description thereon is omitted to avoid redundancy.

When determining that removal of motion is possible in the determination processing of removal of motion (YES in step S34), the control unit 11 sets a sampling frequency of the ADC 110 to a relatively high frequency (high sampling frequency) where motion component is considered (step S35).

Thereafter, the control unit 11 causes the pulse wave sensor 19 to execute pulse wave measurement. The control unit 11 further sets filter for the pulse wave signal output from the pulse wave sensor 19 to the motion removing filter and performs the motion removing filter processing (step S38).

That is, the control unit 11 performs the motion removing filter processing for removing the motion component in order to extract a pulse component from the pulse wave signal waveform superimposed with the motion component.

Note that, when determining that the low power mode is set in the determination processing of low power mode of step S33 (YES in step S33) and when determining that removal of motion is impossible in the determination processing of removing motion of step S34 (NO in step S34), the control unit 11 turns off the LED 17 (to stop irradiation with light) and stops operation of the pulse wave sensor 19, thereby stopping pulse wave measurement.

Here, operation of the PD 18 may further be stopped.

FIGS. 9A and 9B are timing charts illustrating an operation state of the second example.

In FIG. 9A, an acceleration signal (motion data) output from the motion sensor 15 is illustrated. In FIG. 9B, a sampling frequency Fs selected by the control unit 11 is illustrated.

According to the second example, when determining that the user is in the motion state, the control unit 11 sets a relatively high sampling frequency. When determining that the user is in the non motion state, the control unit 11 sets a relatively low sampling frequency. This allows for mitigating power consumption in the ADC 110 in the non motion state.

Note that switching the sampling frequency between the non motion state and motion state is performed by detecting motion with the motion sensor 15 and discriminating between the non motion state and motion state from the size of amplitude or characteristics of the waveform of the acceleration signal. For example, when the amplitude of the acceleration signal is greater than a setting value for a certain period of time or more, it may be determined as the motion state (in motion). For example, when the amplitude of the acceleration signal is smaller than the setting value for a certain period of time or more, it may be determined as the non motion state.

Note that, in the non motion state, pulse wave includes substantially no motion component and thus the sampling frequency may be switched to a lowest frequency possible for observing a pulse wave. Especially, when observing only a pulse rate, the sampling frequency required may further be lowered.

On the other hand, in the non motion state, it is preferable to take samples of motion noise also from high bandwidth in order to remove the motion noise and thus the sampling frequency is preferably relatively high. Therefore, a relatively high sampling frequency is set.

In this manner, by discriminating between the non motion state and motion state from motion detected by the motion sensor 15 and setting a sampling frequency with modification to a preferable frequency as appropriate, power consumption of the ADC 110 can be suppressed to a level least required.

Note that the motion removing filter processing is applied to a pulse wave signal acquired with the sampling frequency having been set.

According to the second example, by detecting motion with the motion sensor 15 and modifying and setting a sampling frequency according to the motion with the control unit 11, a low sampling frequency can be set when the user is in the non motion state, thereby allowing for saving power consumption of the ADC 110. As a result, a life of the battery 13 can be prolonged.

Note that it has been described that, in the second example, the control unit 11 includes the ADC 110. However, the ADC 110 may not be included in the control unit 11 but may be connected to the control unit 11 externally, in which case a similar effect can be obtained.

### THIRD EXAMPLE

A pulse rate of a user in motion usually rises rapidly upon starting the motion and drops rapidly upon stopping the motion. The pulse rate also varies according to a level of the motion and has relatively large variations over time. Therefore, when a pulse rate is monitored during motion, the pulse wave measuring device 10 is preferably in operation at all times.

On the other hand, in the non motion state where the motion is not continuous, variations in the pulse rate over time are small. Thus, when a pulse rate is monitored during the non motion state, the pulse wave measuring device 10 may not be in operation at all times.

Therefore, in the third example described below, a configuration is employed where an interval measurement mode for performing pulse wave measurement only intermittently is set in the non motion state, thereby allowing for saving power.

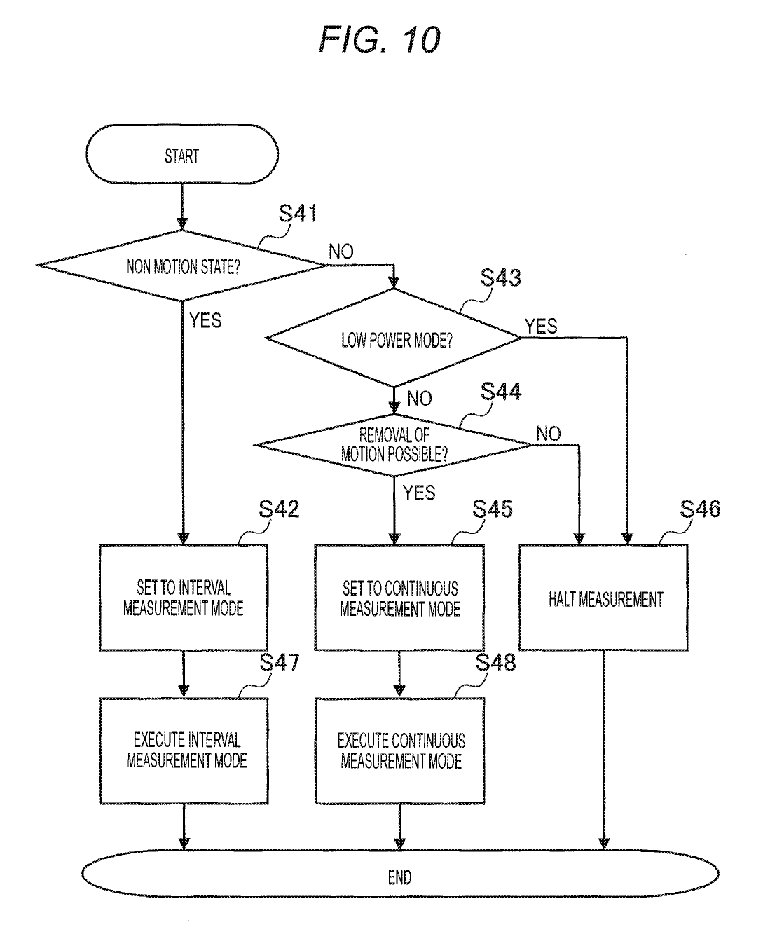

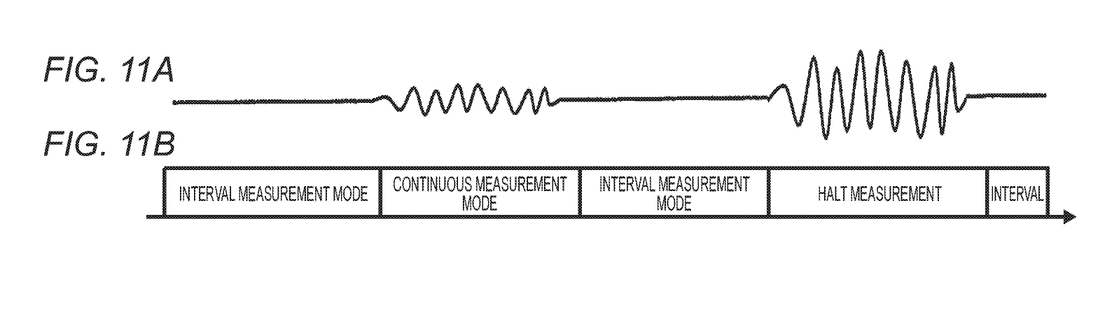

Operations in the third example will be described with reference to a flowchart in FIG. 10 and timing charts in FIGS. 11A and 11B.

In the flowchart in FIG. 10, the control unit 11 first determines whether the user is in the non motion state (step S41).

Details of the determination processing of non motion state is similar to those in the first example where the determination processing is performed based on the acceleration signal (motion signal) from the motion sensor 15.

That is, when the pulse rate is a normal value and a state, where the amplitude of the acceleration signal from the motion sensor 15 is smaller than a setting value, has been maintained during certain period of time, the control unit 11 determines that the user is in the non motion state. Thereafter, a measurement mode is set to the interval measurement mode (step S42).

The pulse wave measuring device 10 repeats measurement operation and a stop with a prescribed time interval in the interval measurement mode.

When the user is in the non motion state, variations in the pulse rate over time are small. Thus, pulse wave measurement is not required to be performed frequently. It is possible to sufficiently capture variations in the pulse rate with pulse wave measurement at intervals.

Therefore, when determining that the user is in the non motion state, the control unit 11 sets a measurement mode to the interval measurement mode where pulse wave measurement is performed intermittently.

Thereafter, the control unit 11 further determines that the motion removing filter processing for removing motion component is not required since substantially no motion component is superimposed to the pulse wave signal in the non motion state. Thereafter, the control unit 11 performs the BPF processing, including a relatively small volume of calculation, to the pulse wave signal (step S47).

Note that, here, the BPF processing may not be performed to the pulse wave signal. The filter processing itself may also be omitted.

On the other hand, when determining that the user is in motion state (NO in step S41), the control unit 11 then determines whether an operation mode of the pulse wave measuring device 10 is set to the low power mode (step S43).

Here, as in the first example, the low power mode allows for operation in a power saving mode with more priority on a battery life and may be manually or automatically set in advance.

When determining that the operation mode is not set to the low power mode (NO in step S43), the control unit 11 then determines whether removal of motion is possible (step S44).

The determination processing of whether removal of motion is possible is similar to the content described in the first example (FIG. 4) and thus description thereon is omitted to avoid redundancy.

When determining that removal of motion is possible in the determination processing of removal of motion (YES in step S44), the control unit 11 sets the continuous measurement mode considering the motion component (step S45).

Thereafter, the control unit 11 performs the motion removing filter processing to the pulse wave signal waveform (step S48).

That is, the control unit 11 performs the motion removing filter processing for removing the motion component in order to extract a pulse component from the pulse wave signal waveform superimposed with the motion component.

Note that, when determining that the low power mode is set in the determination processing of low power mode of step S43 (YES in step S43) and when determining that removal of motion is impossible in the determination processing of removing motion of step S44 (NO in step S44), the control unit 11 turns off the LED 17 (to stop irradiation with light) and stops operation of the pulse wave sensor 19, thereby stopping pulse wave measurement.

Here, operation of the PD 18 may further be stopped.

FIGS. 11A and 11B are timing charts illustrating an operation state of the third example.

In FIG. 11A, an acceleration signal (motion data) output from the motion sensor 15 is illustrated. In FIG. 11B, a measurement mode selected by the control unit 11 is illustrated.

According to the third example, the control unit 11 sets the continuous measurement mode when the user is in the motion state and sets the interval measurement mode when the user is in the non motion state. This allows for suppressing power consumption in the non motion state.

Note that, when a predetermined level of motion is detected or when a predetermined level of pulse rate is detected during the interval measurement mode, the control unit 11 cancels the interval measurement mode and changes to the continuous measurement mode.

According to the third example, the control unit 11 determines whether the user is in the non motion state or motion state from data output from the motion sensor 15. When the user is in the non motion state, the control unit 11 causes the pulse wave measuring device 10 to operate intermittently in the interval measurement mode and when the user is in the motion state the control unit 11 causes the pulse wave measuring device 10 to operate continuously in the continuous measurement mode. This allows for saving power in the non motion state, thereby reducing consumption of the battery 13.

Note that, when an object is to know overall variations in the pulses and thus continuous measurement of pulses is not required, the pulse wave measuring device 10 may be caused to operate intermittently in the continuous motion state. This allows for drastically reducing an average power consumption.

On the other hand, when removal of motion is possible in the motion state in a mode other than the low power mode, the mode is switched to the continuous measurement mode.

In the motion state in the low power mode, or when removal of motion is not possible, pulse wave measurement may be stopped.

Note that power saving by switching filter processing as described in the first example, power saving by switching sampling frequency as described in the second example, and power saving by switching to the interval measurement mode as described in the third example may be combined. In this case, an effect of power saving can be further enhanced as compared to performing one of the above measures individually.

Although the present invention has been described above with the embodiments, it shall be understood that the technical scope of the invention is not limited to the scope described for the above embodiments. It is apparent to those skilled in the art that various modifications or improvements can be applied to the embodiments. It is apparent from the descriptions of claims that an embodiment including such modifications or improvements is also within the technical scope of the invention.

What is claimed is:

1. A biological information measuring device, comprising:
a biological information measuring unit configured to measure biological information of a user;
a motion signal output unit configured to output a motion signal corresponding to motion of the user; and
a control unit,
wherein the control unit causes the biological information measuring unit to measure the biological information when amplitude of the motion signal is smaller than a first setting value during a first period, and
wherein the control unit causes the biological information measuring unit to stop measurement of the biological information when the amplitude of the motion signal is greater than the first setting value during a second period and when there is a period where the amplitude of the motion signal is greater than a second setting value, which is greater than the first setting value, in the second period.

2. The biological information measuring device according to claim 1,
wherein the biological information measuring unit is a pulse wave sensor configured to measure a pulse wave of the user.

3. The biological information measuring device according to claim 1,
wherein the control unit causes the biological information measuring unit to measure the biological information when the amplitude of the motion signal is greater than the first setting value during the second period; the amplitude of the motion signal in the second period is smaller than the second setting value; and the motion signal in the second period has a predetermined periodicity.

4. The biological information measuring device according to claim 3,
wherein the control unit causes the biological information measuring unit to stop measurement of the biological information when the amplitude of the motion signal is greater than the first setting value during the second period; the amplitude of the motion signal in the second period is smaller the second setting value; and the motion signal in the second period does not have the periodicity.

5. The biological information measuring device according to claim 3,
wherein the biological information measuring unit outputs a biological information signal corresponding to the biological information, and
the control unit controls to perform processing by motion removing filter for removing a motion component attribute to the motion of the user in the biological information signal output from the biological information measuring unit when the amplitude of the motion signal is greater than the first setting value during the second period and the biological information measuring unit is caused to measure the biological information.

6. The biological information measuring device according to claim 5,
wherein the control unit controls not to perform processing by the motion removing filter to the biological information signal when the amplitude of the motion signal is smaller than the first setting value during the first period and the biological information measuring unit is caused to measure the biological information.

7. The biological information measuring device according to claim 1,
wherein the biological information measuring unit outputs a biological information signal corresponding to the biological information,
the control unit comprises a conversion unit configured to convert the biological information signal into a digital signal by importing the biological information signal at a timing corresponding to a sampling frequency,
the control unit sets the sampling frequency as a first frequency when the amplitude of the motion signal is smaller than the first setting value during the first period, and
the control unit sets the sampling frequency as a second frequency, which is higher than the first frequency, when the state where the amplitude of the motion signal is greater than the first setting value during the second period.

8. The biological information measuring device according to claim 1,
wherein the control unit sets the biological information measuring unit in an interval measurement mode where the biological information is measured intermittently when the amplitude of the motion signal is smaller than the first setting value during the first period, and
the control unit sets the biological information measuring unit in continuous measurement mode where the biological information is measured continuously when the amplitude of the motion signal is greater than the first setting value during the second period.

9. A biological information measuring device, comprising:
a biological information measuring unit configured to measure biological information of a user;
a motion signal output unit configured to output a motion signal corresponding to motion of the user; and
a control unit configured to control the biological information measuring unit and the motion signal output unit,
wherein the control unit determines that the user is in a motion state when amplitude of the motion signal is greater than a first setting value during a predetermined period which includes a predetermined time length,
the control unit causes the biological information measuring unit to stop measurement of the biological information when it is determined that the user is in the motion state and the amplitude of the motion signal is greater than a second setting value, which is greater than the first setting value, has existed in the predetermined period, and
the control unit controls the biological information measuring unit to measure the biological information when it is determined that the user is in the motion state; the amplitude of the motion signal during the predetermined period is smaller than the second setting value; and the motion signal during the predetermined period has a predetermined periodicity.

10. A driving control method of a biological information measuring device,
wherein the biological information measuring device includes a biological information measuring unit configured to measure biological information of a user, the driving control method comprising the steps of:
causing the biological information measuring unit to measure biological information of the user when amplitude of a motion signal corresponding to motion of the user is smaller than a first setting value during a first period; and
causing the biological information measuring unit to stop measurement of the biological information when the amplitude of the motion signal is greater than the first setting value during a second period and when there is a period where the amplitude of the motion signal is greater than a second setting value, which is greater than the first setting value, in the second period.

11. The driving control method of driving a biological information measuring device according to claim 10, the method comprising the step of:
causing the biological information measuring unit to measure the biological information when the amplitude of the motion signal is greater than the first setting value during the second period; the amplitude of the motion signal in the second period is smaller the second setting value; and the motion signal in the second period has a predetermined periodicity.

12. The driving control method of driving a biological information measuring device according to claim 11, the method comprising the step of:
causing the biological information measuring unit to stop measurement of the biological information when the amplitude of the motion signal is greater than the first setting value during the second period; the amplitude of the motion signal in the second period is smaller than the second setting value; and the motion signal in the second period does not have the periodicity.

13. The driving control method of driving a biological information measuring device according to claim 11,
wherein the biological information measuring unit outputs a biological information signal corresponding to the biological information, and
the method comprises the step of controlling to perform processing by motion removing filter for removing a motion component attributable to motion of the user in the biological information signal output from the biological information measuring unit when the amplitude of the motion signal is greater than the first setting value during the second period and the biological information measuring unit is caused to measure the biological information.

14. The driving control method of driving a biological information measuring device according to claim 13, the method comprising the step of:
controlling not to perform processing by the motion removing filter to the biological information signal when the amplitude of the motion signal is smaller than the first setting value during the first period and the biological information measuring unit is caused to measure the biological information.

15. The driving control method of driving a biological information measuring device according to claim 10,
wherein the biological information measuring unit outputs a biological information signal corresponding to the biological information,
the biological information measuring device comprises a conversion unit configured to convert the biological information signal into a digital signal by importing the biological information signal at a timing corresponding to a sampling frequency, and
the method comprises the steps of:
setting the sampling frequency as a first frequency when the amplitude of the motion signal is smaller than the first setting value during the first period; and
setting the sampling frequency as a second frequency, which is higher than the first frequency, when the amplitude of the motion signal is greater than the first setting value during the second period.

16. The driving control method of driving a biological information measuring device according to claim 10, the method comprising the steps of:
setting at least the biological information measuring unit in an interval measurement mode where the biological information is measured intermittently when the amplitude of the motion signal is smaller than the first setting value during the first period; and
setting at least the biological information measuring unit in a continuous measurement mode where the biological information is measured continuously when the amplitude of the motion signal is greater than the first setting value during the second period.
